(19) United States
(12) Patent Application Publication
(10) Pub. No.: US 2025/0146041 A1
(43) Pub. Date: May 8, 2025

(54) METHODS FOR CELL FREE PROTEIN SYNTHESIS AND POST TRANSLATIONAL MODIFICATION OF THE EXPRESSED PROTEINS

(71) Applicant: Nuclera LTD, Cambridge (GB)

(72) Inventors: Michael Chun Hao Chen, Cambridge (GB); Gordon Ross Mclnroy, Cambridge (GB)

(21) Appl. No.: 18/837,628

(22) PCT Filed: Feb. 13, 2023
(86) PCT No.: PCT/GB2023/050325

§ 371 Date: Aug. 12, 2024

(30) Foreign Application Priority Data
Feb. 14, 2022 (GB) 2201896.4
Aug. 19, 2022 (GB) 2212126.3

(51) Int. Cl. C12P 21/06; C12N 9/50

(52) U.S. Cl. CPC C12P 21/06; C12N 9/506; C12Y 304/22028

(57) ABSTRACT

Provided herein are methods of cell-free protein synthesis, optimised cell-free protein synthesis (CFPS) reagents and the post translational modification of the expressed proteins, to increase protein expression yields. The methods are applicable to protein expression on a microfluidic device having hydrophobic surfaces by merging droplets on the device in order to screen a selection of expression and post translational modification compositions in parallel.

### FIELD OF THE INVENTION

Provided herein are methods of cell-free protein synthesis and post translational modification, optimised cell-free protein synthesis (CFPS) reagents, and methods for optimising CFPS reagents to increase protein expression yields and post translational modification. In certain embodiments the methods are applicable to protein expression on a microfluidic device having hydrophobic surfaces.

### BACKGROUND TO THE INVENTION

Cell-free protein synthesis (CFPS) has become an important tool for molecular biologists by playing a central role in a wide variety of applications. Cell-free systems can be categorized into two main classes: cell extracts and recombinant systems. Cell extracts are highly functional but complex and undefined systems. In 2001, Shimizu et al. demonstrated that a defined cell-free system called the “PURE” system (protein synthesis using recombinant elements) could be reconstituted from purified recombinant components.

The biggest advantage of CFPS is that it is the quickest way to obtain an expressed phenotype (protein) from a genotype (gene). Starting with a PCR or plasmid template, in-vitro protein synthesis and functional assays can be carried out in a few hours. Moreover, it is independent of host cells. However, extract-based systems are known to often contain nonspecific nucleases and proteases that adversely affect protein synthesis. CFPS systems are open systems that are suitable for modification by addition of external components.

Protein produced using CFPS can be modified after translation. Typical transformations may include cleavage of the protein to remove tags, glycosylation, acetylation or phosphorylation. Prior art methods to modify proteins after translation typically involve addition of reagents to the purified protein. Protein may be translated in the presence of agents which help with folding, such as chaperones, but these to not covalently modify the proteins after translation.

To date, digital microfluidics, electrowetting-on-dielectric (EWoD), and electrokinesis in general have only found limited uses in cell-free biological-based applications, mostly due to biofouling, where biological components such as proteins, nucleic acids, crude cell extracts and other bioproducts adsorb and/or denature to hydrophobic surfaces. Biofouling is well known in the art to limit the ability of EWoD devices to manipulate droplets containing biomacromolecules. Wheeler and colleagues report that the maximum actuation time for droplets on EWoD devices containing biological media is 30 min before biofouling inhibits EWoD-based droplet actuation (Langmuir 2011, 27, 13, 8586-8594).

On an EWoD device, cell lysate expression leads to variable outcomes. The level of protein expression in different droplets on the same device is not consistent. The use of purified component mixtures gives reliable expression, but at a lower level than the best cell lysate mixtures. The inventors herein have improved the reliability of expression levels of proteins. Additionally, purified component mixtures are typically not capable of performing downstream maturation of the produced protein into the desired form. For example, purified component mixtures are not capable of effecting post-translational modification of proteins, such as glycosylation. The inventors herein have improved the utility of purified component mixtures for CFPS.

Posttranslational modifications (PTMs) are covalent processing events that change the molecular weight of a protein by proteolytic cleavage or adding a modifying group, such as acetyl, phosphoryl, glycosyl and methyl, to one or more amino acids. PTMs play a key role in numerous biological processes by significantly affecting the structure and dynamics of proteins. PTMs can occur in a single type of amino acid or multiple amino acids and lead to changes in the chemical properties of modified sites. PTMs are seen in many proteins such as secretory proteins, membrane proteins and histones. The modifications affect a wide range of protein behaviours and characteristics, including enzyme function and assembly, protein lifespan, protein-protein interactions, cell-cell and cell-matrix interactions, molecular trafficking, receptor activation, protein solubility, protein folding and protein localization. Therefore, these modifications are involved in various biological processes such as signal transduction, gene expression regulation, gene activation, DNA repair and cell cycle control.

### SUMMARY

Disclosed herein is a method of synthesizing a protein, comprising using a reaction system comprising:
a. at least one template nucleic acid encoding a protein of interest; and
b. a cell-free protein synthesis reagent;
wherein the ratios of components present in b. have been optimised to increase the yield of protein expression.

Disclosed herein is a method of synthesizing a protein comprising using a reaction system having:
a. at least one template nucleic acid encoding a protein of interest,
b. a reagent composition having enzymes for protein synthesis wherein the composition is a cell lysate which has been supplemented with additional purified protein components.

The protein synthesis reaction reagent can be a mixture of cell lysate and purified components, for example a system of purified recombinant elements i.e. protein synthesis using recombinant elements (PURE).

Particularly the enzymes used for protein expression can be a mixture of cell lysates and purified enzymes.

The protein synthesis reaction reagent may comprise:
i. synthesized or isolated ribosomes, initiation factors, elongation factors, termination factors, aminoacyl-tRNA synthetases, methionyl tRNA transformylases, tRNAs, amino acids, ribonucleoside triphospates, 10-formyl 5,6,7,8-tetrahyrofolic acid (FD), salts and water.

Disclosed is a method for expressing and changing the molecular weight of an expressed protein using a single reagent composition comprising a template nucleic acid, reagents for cell-free protein synthesis and one or more enzymes which post translationally covalently modify and amend the molecular weight of the expressed protein.

The synthesis may be performed in a microfluidic device, for example an electrowetting-on-dielectric (EWoD) device. Alternatively the synthesis may be performed in a microtitre plate format.

Disclosed herein is a method for the synthesis of a protein on an electrowetting-on-dielectric (EWoD) device, the method comprising taking a reaction system having at least one template nucleic acid encoding a protein of interest, blending droplets to form a series of droplets having varying cell-free reagent compositions, including enzymes for protein synthesis and the template nucleic acid and monitoring the synthesis of the protein of interest in the various compositions, thereby identifying a composition suitable for expression of the protein of interest.

The reaction system may also comprise additional components that increase the yield of protein expression. The additional components may be selected from the following: cell lysates derived from naturally occurring or engineered cell lines, additional protein components, or chemical entities. The chemical entities may be one or more of a polyethylene glycol, an allolactose, an aldohexose or a thiogalactopyranoside.

Where the synthesis is performed on a microfluidic device, the lysate mixture can be optimised by combining reagents on the device. For example concentrations of reagents that give optimal expression can be identified by blending components at different ratios in a large number of droplets and the expression monitored in parallel to identify optimal compositions.

An aspect of the invention includes an improved system for synthesizing a protein, the system having:
a. at least one template nucleic acid encoding a protein of interest; and
b. a cell-free protein synthesis reagent;
   wherein the cell-free protein synthesis reagent contains synthesized or isolated ribosomes, initiation factors, elongation factors, termination factors, aminoacyl-tRNA synthetases, methionyl tRNA transformylases, tRNAs, amino acids, ribonucleoside triphospates, 10-formyl 5,6,7,8-tetrahyrofolic acid (FD), salts and water and further contains one or more additional components that improves the expression on an EWoD device. The additional components may be selected from metal ions, co-factors, chaperones, reducing agents, polyethylene glycol, an allolactose, an aldohexose or a thiogalactopyranoside.

The cell-free protein synthesis reagents can include an additional component being a cell lysate. The cell lysate contains a mixture of proteins and other reagents obtained from a cell without purification or separation of particular proteins.

The cells lysates may be derived from mammalian cells, prokaryotic cells, yeast cells, plant cells or protozoa. The cell lysates may be derived from human embryonic kidney cells (HEK293), Chinese hamster ovary cells (CHO), HeLa, BHK21, NS0, or Sp2/0 cells. The cell lysates may be derived from Escherichia coli cells, Saccharomyces cerevisiae or Pichia pastoris cells, tobacco or wheat cells, or Leishmania tarentolae.

Using only cell lysates as the reaction reagent has shown to give variable and unreliable protein expression yield. Whereas, using only purified components as the reaction reagent gives a lower protein expression yield but with more consistent results. Combining cell lysates and PURE reagents within the reaction reagent for protein expression has shown a decrease in variability and a more consistent protein expression yield.

The in-vitro transcription and translation may be coupled or uncoupled.

The expressed protein may be fused to a peptide tag. The peptide tag may be one component of a fluorescent protein and the further polypeptide a complementary portion of the fluorescent protein. The fluorescent protein could include sfGFP, ccGFP, GFP, eGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide NFAST in the presence of a hydroxybenzylidene rhodanine analog.

For example, the GFP₁₋₁₀ polypeptide amino acid sequence could be derived from sfGFP:

SEQ ID NO: 1
MSKGEELFTGVVPILVELDGDVNGHKFSVRGEGEGDATNGKLTLKFICTT
GKLPVPWPTLVTTLTYGVQCFSRYPDHMKRHDFFKSAMPEGYVQERTISF
KDDGTYKTRAEVKFEGDTLVNRIELKGIDFKEDGNILGHKLEYNFNSHNV
YITADKQKNGIKANFKIRHNVEDGSVQLADHYQQNTPIGDGPVLLPDNHY
LSTQSVLSKDPNEK

Alternatively, the GFP₁₋₁₀ polypeptide amino acid sequence could be further mutated from the sequence above to become brighter more quickly upon complementation. The sequence may have a greater than 90% homology to any sequence mentioned herein. The sequence may have a greater than 95% homology to any sequence mentioned herein.

SEQ ID NO: 2
MSKGEELFTGVVPILVELDGDVNGHKFSVRGEGEGDATIGKLTLKFICTT
GKLPVPWPTLVTTLTYGVQCFSRYPDHMKRHDFFKSAMPEGYVQERTISF
KDDGKYKTRAVVKFEGDTLVNRIELKGTDFKEDGNILGHKLEYNFNSHNV
YITADKQKNGIKANFTVRHNVEDGSVQLADHYQQNTPIGDGPVLLPDNHY
LSTQTVLSKDPNEK

The GFP₁₋₁₀ polypeptide amino acid sequence could also be derived from ccGFP, having a greater than 90 or 95% homology to:

(ccGFP₁₋₁₁)
SEQ ID NO: 3
MSLSKQVVKEDMKMTYHMDGCVNGHYFTIEGEGTGKPFKGQKTLKLRVTEGGPLPFAFDI
LSATFTYGNRCFCDYPEDMPDYFKQSLPEGYSWERTMMYEDGACGTASAHISLDKNGFV
HNSTFHGVNFPANGPVMKKKGVNWEPSSEKITACDGILKGDVTMFLVLEGGHRLKCLFQTT
YKADKVVKMPPNHIIEHRLVRSEDGDAVQI QEHAVAKYFTV
(ccGFP₁₋₁₀)
SEQ ID NO: 4
MSLSKQVVKEDMKMTYHMDGCVNGHYFTIEGEGTGKPFKGQKTLKLRVTEGGPLPFAFDI
LSATFTYGNRCFCDYPEDMPDYFKQSLPEGYSWERTMMYEDGACGTASAHISLDKNGFV
HNSTFHGVNFPANGPVMKKKGVNWEPSSEKITACDGILKGDVTMFLVLEGGHRLKCLFQTT
YKADKVVKMPPNHIIEHRLVRSED
(ccGFP₁₋₁₀)
SEQ ID NO 5
MSMEKQVLKENMKTTYHMDGSVDGHYFEIEGEGTGNPFKGEQELKLRVTKGGPLPFAFDI
LSPTFTYGNRVFTDYPEDMPDYFKQSLPEGYSWERTMMYEDGATATASARISLDKNGFVH
KSTFHGENFPANGPVMKKKGVDWEPSSETITPEDGILKGDVEMFLVLEGGQRLKALFQTTY
KANKVVKMPPRHKIEHRLVRS
Nucleic acid sequence to express seq ID No 5 ccGFP₁₋₁₀;
SEQ ID NO: 6
5′atgagcatggaaaaacaggtgctgaaagaaaacatgaaaaccacctatcacatggatggtagcgttg
atggtcactattttgaaattgaaggtgaaggcaccggcaatccgtttaaaggtgaacaagaactgaaac
tgcgtgttaccaaaggtggtccgctgccgtttgcatttgatattctgagcccgacctttacctatggta
atcgtgtttttaccgactatccggaagatatgccggattatttcaaacagagcctgccggaaggttata
gctgggaacgtaccatgatgtatgaagatggtgcaaccgcaaccgccagcgcacgtattagcctggata
aaaatggttttgtgcataagagcacctttcacggtgaaaactttccggcaaatggtccggttatgaaaa
agaaaggtgttgattgggaaccgagcagcgaaaccattacaccggaagatggtattctgaaaggtgatg
ttgaaatgtttctggttctggaaggtggtcagcgtctgaaagccctgtttcagaccacctataaagcca
ataaagtggttaaaatgcctccgcgtcataaaattgaacatcgtctggttcgtagc
SEQ ID NO 7
MSMSKQVLKENMKTTYHMDGSVNGHYFTIEGEGTGNPFKGQQSLKLRVTKGGPLPFAFDI
LSPTFTYGNRVFTDYPEDMPDYFKQSLPEGYSWERTMMYEDGATATASARISLDKNGFVH
KSTFHGENFPANGPVMKKKGVNWEPSSETITPSDGILKGDVTMFLVLEGGQRLKALFQTTY
KANKVVKMPPRHKIEHRLVRS
Nucleic acid sequence to express seq ID No 7 ccGFP₁₋₁₀;
SEQ ID NO: 8
5′atgagcatgagcaaacaggtgctgaaagaaaatatgaaaaccacctatcacatggatggtagcgtta
atggtcactattttaccattgaaggtgaaggcaccggtaatccgtttaaaggtcagcagagcctgaaac
tgcgtgttaccaaaggtggtccgctgccgtttgcatttgatattctgagcccgacctttacctatggta
atcgtgtttttaccgactatccggaagatatgccggattatttcaaacagagcctgccggaaggttata
gctgggaacgtaccatgatgtatgaagatggtgcaaccgcaaccgccagcgcacgtattagcctggata
aaaatggttttgtgcataagagcacctttcacggtgaaaactttccggcaaatggtccggttatgaaaa
agaaaggtgttaattgggaaccgagcagcgaaaccattacaccgagtgatggtattctgaaaggtgatg
ttaccatgtttctggttctggaaggtggtcagcgtctgaaagccctgtttcagaccacctataaagcca
ataaagtggttaaaatgcctccgcgtcataaaattgaacatcgtctggttcgtagc

The complementary GFP₁₁ peptide amino acid sequence could be the following:

| | (SEQ ID NO: 9) |
|---|---|
| 1. | KRDHMVLLEFVTAAGITGT |
| | (SEQ ID NO: 10) |
| 2. | KRDHMVLHEFVTAAGITGT |
| | (SEQ ID NO: 11) |
| 3. | KRDHMVLHESVNAAGIT |
| | (SEQ ID NO: 12) |
| 4. | RDHMVLHEYVNAAGIT |
| | (SEQ ID NO: 13) |
| 5. | GDAVQIQEHAVAKYFTV |
| | (SEQ ID NO: 14) |
| 6. | GDTVQLQEHAVAKYFTV |
| | (SEQ ID NO: 15) |
| 7. | GETIQLQEHAVAKYFTE |

GFP₁₁ or GFP₁₋₁₀ can be fused to the protein of interest through an amino acid linker. In one embodiment, the oligopeptide, peptide, or polypeptide linker can be 0-50 amino acids.

Also disclosed are nucleic acid sequences for expressing particular tags. Nucleic acid sequences include

SEQ ID NO: 16
5′GGTGATACCGTTCAGCTGCAAGAACATGCAGTTGCAAAATACTTTAC
CGTG
SEQ ID NO: 17
5′GGTGAAACCATCCAGTTACAAGAACACGCCGTGGCCAAATATTTCAC
CGAA

These sequences may be repeated one or more times to produce a protein having multiple GFP₁₁ domains.

For example, the sfCherry₁₋₁₀ polypeptide amino acid sequence could be:

SEQ ID NO: 18
MEEDNMAIIKEFMRFKVHMEGSVNGHEFEIEGEGEGHPYEGTQTAKLKV
TKGGPLPFAWDILSPQFMYGSKAYVKHPADIPDYLKLSFPEGFTWERVM
NFEDGGVVTVTQDSSLQDGEFIYKVKLLGTNFPSDGPVMQKKTMGWEAS
TERMYPEDGALKGEINQRLKLKDGGHYDAEVKTTYKAKKPVQLPGAYNV
DIKLDITSHNED

The complementary sfCherry11 peptide amino acid sequence could be:

SEQ ID NO: 19
YTIVEQYERAEGRHSTGG

sfCherry11 or sfCherry₁₋₁₀ can be fused to the protein of interest through an amino acid linker. In one embodiment, the oligopeptide, peptide, or polypeptide linker can be 0-50 amino acids.

For example, the NFAST polypeptide amino acid sequence could be:

SEQ ID NO: 20
MEHVAFGSEDIENTLAKMDDGQLDGLAFGAIQLDGDGNILQYNAAEGDI
TGRDPKQVIGKNFFKDVAPGTDSPEFYGKFKEGVASGNLNTMFEWMIPT
SRGPTKVKVHMKKALS

The complementary CFAST11 peptide amino acid sequence could be:

SEQ ID NO: 21
GDSYWVFVKRV

Or the complementary CFAST10 peptide amino acid sequence could be:

SEQ ID NO: 22
GDSYWVFVKR

NFAST, CFAST₁₁, and/or CFAST₁₀ can be fused to the protein of interest through an amino acid linker. In one embodiment, the oligopeptide, peptide, or polypeptide linker can be 0-50 amino acids.

The peptide tag may also be one component of a protein that forms a detectable substrate, such as a luminescent or colorigenic substrate. The protein could include beta-galactosidase, beta-lactamase, or luciferase.

The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry₁₁ peptide tags and the synthesis occurs in the presence of multiple sfCherry₁₋₁₀ polypeptides. The protein of interest may be fused to one or more sfCherry₁₁ peptide tags and one or more GFP₁₁ peptide tags and the synthesis occurs in the presence of one or more GFP₁₋₁₀ polypeptides and one or more sfCherry₁₋₁₀ polypeptides.

Any protein of interest may be synthesised. The protein may be an enzyme, for example a terminal deoxynucleotidyl transferase (TdT) enzyme or a truncated version thereof or the homologous amino acid sequence of a terminal deoxynucleotidyl transferase (TdT) enzyme in other species or the homologous amino acid sequence of Polμ, Polβ, Polλ, and Polθ of any species or the homologous amino acid sequence of X family polymerases of any species.

### FIGURES

FIG. 1 shows the fluorescence signal from a 3C cleavage experiment on an electrowetting device.

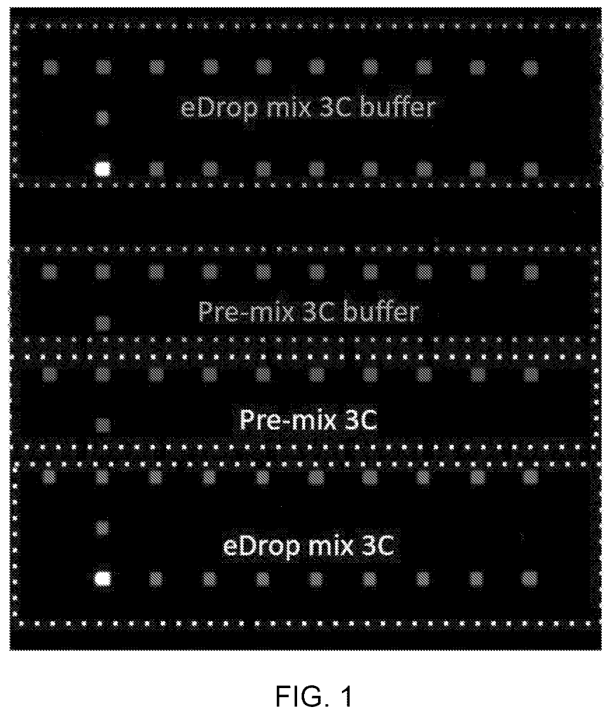

FIG. 2 shows expression of VEGF protein in the presence of 3C protease and that 3C cleavage happens on the electrowetting device, showing SDS-PAGE analysis of complemented and 3C cleaved HIS_ccGFP₁₁:ccGFP₁₋₁₀-MBP. The full length protein with the complemented ccGFP₁₋₁₀ is at ˜110 kDa (SUMO_VEGF_3C-His-ccGFP₁₁:ccGFP₁₋₁₀-MBP). The band at ˜70 kDa (3C-His-ccGFP₁₁:ccGFP₁₋₁₀-MBP) is the cleaved product 3C-His-ccGFP₁₁:ccGFP₁₋₁₀-MBP. SUMO_VEGF cleaved protein at ˜45 kDa.

FIG. 3 shows plate reader measurements which indicated that the total protein detected in absence of 3C had lower fluorescent yield when the detector is bound to the protein VEGF with solubility tag. In the presence of 3C protease, when complemented after 3C cleavage of SUMO_VEGF, the total and soluble fraction were detected at higher yields.

FIG. 4 shows the image from an electrowetting device with droplets with or without 3C protease. The image below shows the fluorescence signal from 10 hours expression and 5 hours complementation.

FIG. 5 shows the protein constructs made for FIG. 4.

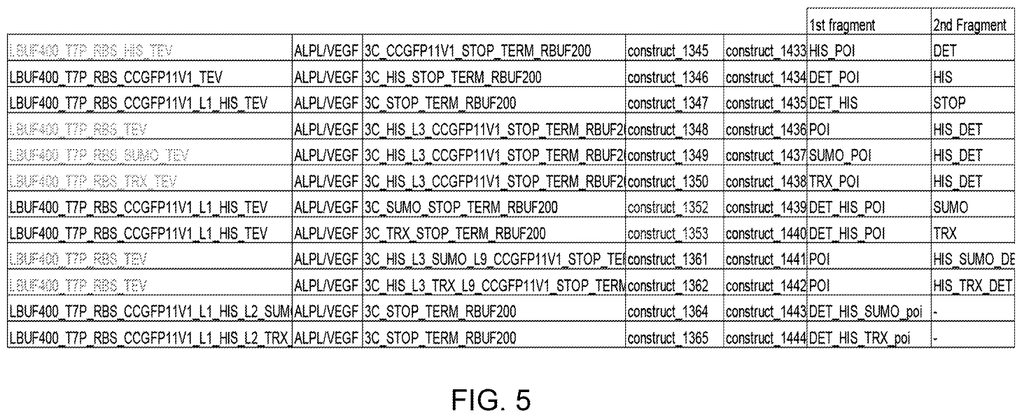

### DETAILED DESCRIPTION OF THE INVENTION

Protein produced using CFPS can be modified after translation. Typical transformations may include cleavage of the protein to remove tags, glycosylation, acetylation or phosphorylation. Prior art methods to modify proteins after translation typically involve addition of reagents to the purified protein. Protein may be translated in the presence of agents which help with folding, such as chaperones, but these to not covalently modify the proteins after translation.

Disclosed herein are methods for expressing and changing the molecular weight of an expressed protein using a single reagent composition comprising a template nucleic acid, reagents for cell-free protein synthesis and one or more enzymes which post translationally covalently modify and amend the molecular weight of the expressed protein.

The single composition performs both translation and post-translational modification. The modification involves a covalent change to the protein strands, not just folding/refolding or disulfide bond formation. The protein changes molecular weight after expression and folding has taken place and does not re-fold the expressed protein.

Disclosed herein is a method of synthesizing a protein, comprising using a reaction system comprising:
a. at least one template nucleic acid encoding a protein of interest; and
b. a protein synthesis reaction reagent;
wherein, the ratios of components present in b. have been optimised to increase the yield of protein expression.

Disclosed is a method of synthesising a protein in a digital microfluidic device. The droplets having the components required for cell-free protein synthesis (CFPS), otherwise known as in-vitro protein synthesis, can be manipulated by electrokinesis in order to effect and improve protein expression.

Protein expression is dependent on the conditions and reagents used for expression. The best expression system for a given protein of interest is not predictable, and may require screening of a large number of similar conditions in order to identify the optimal expression system. The use of EWoD devices can screen large numbers of closely related conditions in parallel in droplets on the device. The droplets can be blended on the device in order to prepare the reagents. For example a cell lysate or reconstituted protein system or mix thereof can be supplemented with a variety of additional components at a selection of concentrations. For example a salt screen, buffer screen or pH screen can be performed across a range of conditions and at variable concentrations.

Disclosed herein is a method for the synthesis of a protein on an electrowetting-on-dielectric (EWoD) device, the method comprising taking a reaction system having at least one template nucleic acid encoding a protein of interest, blending droplets to form a series of droplets having varying cell-free reagent compositions, including enzymes for protein synthesis and the template nucleic acid and monitoring the synthesis of the protein of interest in the various compositions, thereby identifying a composition suitable for expression of the protein of interest. The blend may be a mixture of cell lysate and reconstituted expression systems.

Electrowetting is the modification of the wetting properties of a surface (which is typically hydrophobic) with an applied electric field. Microfluidic devices for manipulating droplets or magnetic beads based on electrowetting have been extensively described. In the case of droplets in channels this can be achieved by causing the droplets, for example in the presence of an immiscible carrier fluid, to travel through a microfluidic channel defined by the walls of a cartridge or microfluidic tubing. Embedded in the walls of the cartridge or tubing are electrodes covered with a dielectric layer each of which are connected to an A/C biasing circuit capable of being switched on and off rapidly at intervals to modify the electrowetting field characteristics of the layer. This gives rise to the ability to steer the droplet along a given path.

As an alternative to microfluidic channel systems, droplets can also be generated and manipulated on planar surfaces using digital microfluidics (DMF). In contrast to channel based microfluidics, DMF utilizes alternating currents on an electrode array for moving fluid on the surface of the array. Liquids can thus be moved on an open-plan device by electrowetting. Digital microfluidics allows precise control over the droplet movements including droplet fusion and separation.

Cell-free protein synthesis, also known as in-vitro protein synthesis or CFPS, is the production of peptides or proteins using biological machinery in a cell-free system, that is, without the use of living cells. The in-vitro protein synthesis environment is not constrained within a cell wall or limited by conditions necessary to maintain cell viability, and enables the rapid production of any desired protein from a nucleic acid template, usually plasmid DNA or RNA from an in-vitro transcription. CFPS has been known for decades, and many commercial systems are available. Cell-free protein synthesis encompasses systems based on crude lysate (Cold Spring Harb Perspect Biol. 2016 December; 8(12): a023853) and systems based on reconstituted, purified molecular reagents, such as the PURE system for protein production (Methods Mol Biol. 2014; 1118:275-284). CFPS requires significant concentrations of biomacromolecules, including DNA, RNA, proteins, polysaccharides, molecular crowding agents, and more (Febs Letters 2013, 2, 58, 261-268).

To date, digital microfluidics, electrowetting-on-dielectric (EWoD), and electrokinesis in general have only found limited uses in cell-free biological-based applications, mostly due to biofouling, where biological components such as proteins, nucleic acids, crude cell extracts and other bioproducts adsorb and/or denature to hydrophobic surfaces. Biofouling is well known in the art to limit the ability of EWoD devices to manipulate droplets containing biomacromolecules. Wheeler and colleagues report that the maximum actuation time for droplets on EWoD devices containing biological media is 30 min before biofouling inhibits EWoD-based droplet actuation (Langmuir 2011, 27, 13, 8586-8594).

Digital microfluidics can be carried out in an air-filled system where the liquid drops are manipulated on the surface in air. However, at elevated temperatures or over prolonged periods, the volatile aqueous droplets simply dry onto the surface by evaporation. This issue is compounded by the high surface area to volume ratio of nanoliter and microliter sized drops. Hence air-filled systems are generally not suitable for protein expression where the temperature of the system needs to be maintained at a temperature suitable for enzyme activity and the duration of the synthesis needs to be prolonged for synthesized protein levels to be detectable.

Protein expression typically requires an ample supply of oxygen. The most convenient and high yielding way to power CFPS is via oxidative phosphorylation where O₂ serves as the final electron acceptor; however, there are other ways that involve replenishing with energy molecules not involved in oxidative phosphorylation. In a confined microfluidic or digital microfluidic system of droplets, insufficient oxygen is available to enable efficient protein synthesis.

The requirement for oxygen is highest when a cellular lysate system is used for expression. Reconstituted systems do not require as much oxygen and do not consume oxygen via metabolic pathways. For expression in sealed droplets where hypoxia may be a problem, blending of the two expression systems is advantageous as the protein expression levels are consistent and metabolic oxidation is reduced.

Described herein are improved methods allowing for the cell-free expression of peptides or proteins in a digital microfluidic device. Included is a method for the cell-free expression of peptides or proteins in a microfluidic device wherein the method comprises one or more droplets containing a nucleic acid template (i.e., DNA or RNA) and a cell-free system having components for protein expression in an oil-filled environment, and moving said droplets using electrokinesis. The components for the cell-free protein synthesis droplet can be pre-mixed prior to introduction to or mixed on the digital microfluidic device. The components for the cell-free protein synthesis droplet can comprise both reconstituted systems (PURE reagents) and cell lysates.

The droplet can be repeatedly moved for at least a period of 30 minutes whilst the protein is expressed. The droplet can be repeatedly moved for at least a period of two hours whilst the protein is expressed. The droplet can be repeatedly moved for at least a period of twelve hours whilst the protein is expressed. The act of moving the droplet allows oxygen to be supplied to the droplet and dispersed throughout the droplet. The act of moving improves the level of protein expression over a droplet which remains static.

The droplet can be moved using any means of electrokinesis. The droplet can be moved using electrowetting-on-dielectric (EWoD). The electrical signal on the EWoD or optical EWoD device can be delivered through segmented electrodes, active-matrix thin-film transistors, or digital micromirrors.

The filler liquid may be a hydrophobic or non-ionic liquid. For example the filler liquid may be decane or dodecane. The filler fluid may be a silicone oil such as dodecamethylpentasiloxane

(DMPS). The filler liquid may contain a surfactant, for example a sorbitan ester such as Span 85.

The oil in the device can be any water immiscible liquid. The oil can be mineral oil, silicone oil, an alkyl-based solvent such as decane or dodecane, or a fluorinated oil. The oil can be oxygenated prior to or during the expression process. Alternatively, the device can be an air-filled device where droplets containing cell-free protein synthesis reagents are rapidly moved into position and fixed into an array under a humidified gas to prevent evaporation. Humidification can be achieved by enclosing or sealing the digital microfluidic device and providing on-board reagent reservoirs. Additionally, humidification can be achieved by connecting an aqueous reservoir to an enclosed or sealed digital microfluidic device. The aqueous reservoir can have a defined temperature or solute concentration in order to provide specific relative humidities (e.g., a saturated potassium sulfate solution at 30° C.).

A source of supplemental oxygen can be supplied to the droplets. For example droplets or gas bubbles containing gaseous or dissolved oxygen can be merged with the droplets during the protein expression. Additionally, a source of supplemental oxygen can be found by oxygenating the oil that is used as the filler medium. It is well-known in the art that oils such as hexadecane, HFE-7500, and others can be oxygenated to support the oxygen requirements of cell growth, especially E. coli cell growth (RSC Adv., 2017, 7, 40990-40995). Oxygenation can be achieved by aerating the oil with pure oxygen or atmospheric air.

The droplets can be formed before entering the microfluidic device and flowed into the device. Alternatively the droplets can be merged on the device. Included is a method comprising merging a first droplet containing a nucleic acid template such as a plasmid with a second droplet containing a cell-free extract having the components for protein expression to form a combined droplet capable of cell-free protein synthesis.

The droplets can be split on the device either before or after expression. Included herein is a method further comprising splitting the aqueous droplet into multiple droplets. If desired the split droplets can be screened with further additives. Included is a method wherein one or more of the split droplets are merged with additive droplets for screening.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

In order to optimise expression, the expression system can be supplemented with additional components, including purified enzymes. The additional components may include salts, co-factors, buffers, surfactants, chaperones or additional protein components. The additional protein components may be selected from for example chaperones, glycosylating enzymes, proteases, redox active enzymes, phosphorylases and kinases.

The additional protein components may be involved in any post-translational modification process, for example methylation, ubiquitinylation, sumoylation, isoprenylation or glycosylation.

Common post-translation modifications include phosphorylation, methylation, sulfation, acetylation, ubiquitylation, prenylation, myristoylation, SUMOylation, palmitoylation, different types of glycosylation (N-glycosylation, O-glycosylation, C-glycosylation and S-glycosylation), phosphoglycosylation and glycosylphosphatidylinositol (GPI anchored).

Protein phosphorylation is an important reversible regulatory mechanism that plays a key role in the activities of many enzymes, membrane channels and many other proteins in prokaryotic and eukaryotic organisms. Phosphorylation target sites are Ser, Thr, Tyr, His, Pro, Arg, Asp and Cys amino acid residues, but mainly happens on Ser, Thr, Tyr and His residues. Phosphorylation involves transferring a phosphate group from adenosine triphosphate to the receptor residues by kinase enzymes. Conversely, dephosphorylating or removal of a phosphate group is an enzymatic reaction catalyzed by phosphatases. Phosphorylation can change the function of proteins via one of the two principal ways: by allostery or by binding to interaction domains.

Acetylation is typically catalyzed via lysine acetyltransferase (KAT) and histone acetyltransferase (HAT) enzymes. Acetyltransferases use acetyl CoA as a cofactor for adding an acetyl group (COCH₃) to the ε-amino group of lysine side chains, whereas deacetylases (HDACs) remove an acetyl group on lysine side chains. Forms of acetylation include Nα-acetylation, Nε-acetylation and O-acetylation, and may occur on Lys, Ala, Arg, Asp, Cys, Gly, Glu, Met, Pro, Ser, Thr and Val residues with different frequencies, although the acetylation is more reported on Lysine residue. Ne-acetylation is more biologically significant compared to the other types of acetylation. Acetylation has an essential role in biological processes such as chromatin stability, protein-protein interaction, cell cycle control, cell metabolism, nuclear transport and actin nucleation.

Ubiquitylation is an important reversible PTM and can occur on all 20 amino acids, however, it occurs on lysine more frequently. This PTM has a major role in the degradation of intracellular proteins via the ubiquitin (Ub)-proteasome pathway. Ubiquitylation is catalyzed by an enzyme complex that contains ubiquitin-activating (E1), ubiquitin-conjugating (E2) and ubiquitin ligase (E3) enzymes. Ubiquitinated proteins may be acetylated on Lys, or phosphorylated on Ser, Thr or Tyr residues. Ubiquitylation modification in substrate proteins can be removed by several specialized families of proteases called deubiquitinases. Ubiquitination plays important roles in stem cell preservation and differentiation by regulation of the pluripotency, and plays a role in many various cell activities such as proliferation, regulation of transcription, DNA repair, replication, intracellular trafficking and virus budding, the control of signal transduction, degradation of the protein, innate immune signaling, autophagy and apoptosis.

Methylation is a reversible PTM, which often occurs in the cell nucleus and on the nuclear proteins such as histone proteins. Methylation occurs on the Lys, Arg, Ala, Asn, Asp, Cys, Gly, Glu, Gln, His, Leu, Met, Phe and Pro residues in target proteins, with lysine and arginine the two main target residues, at least in eukaryotic cells. One important role of methylation is in histone modification. Histone proteins, after synthesis of their polypeptide chains, are methylated at Lys, Arg, His, Ala or Asn residues. Ne-lysine methylation is one of the most abundant histone modifications in eukaryotic chromatin, involves transferring the methyl groups from S-adenosylmethionine to histone proteins via methyltransferase enzyme. In eukaryotes, methylated arginine has been observed in histone and non-histone proteins. Recent studies have shown that methylation is associated with fine tuning of various biological processes ranging from transcriptional regulation to epigenetic silencing via heterochromatin assembly.

Glycosylation occurs in multiple subcellular locations, such as endoplasmic reticulum, the

Golgi apparatus, cytosol and the sarcolemma membrane. Glycosylation occurs in eukaryotic and prokaryotic membranes and secreted proteins, and nearly 50% of the plasma proteins are glycosylated. In this modification, oligosaccharide chains are linked to specific residues by a covalent bond. This enzymatic process, which is catalyzed by a glycosyltransferase enzyme, usually occurs in the side chain of residues such as Trp, Ala, Arg, Asn, Asp, Ile, Lys, Ser, Thr, Val, Glu, Pro, Tyr, Cys and Gly; however, it occurs more frequently on Ser, Thr, Asn and Trp residues in proteins and lipoproteins. According to the target residues, glycosylation can be classified into six groups: N-glycosylation, O-glycosylation, C-glycosylation, S-glycosylation, phosphoglycosylation and glypiation (GPI-anchored). N-glycosylation and O-glycosylation are two major types of glycosylation and have important roles in the maintenance of protein conformation and activity. Glycosylation has a great role in many important biological processes such as cell adhesion, cell-cell and cell-matrix interactions, molecular trafficking, receptor activation, protein solubility effects, protein folding and signal transduction, protein degradation, and protein intracellular trafficking and secretion.

Small Ubiquitin-Related Modifier (SUMO) ylation has been discovered in a wide range of eukaryotic organisms. SUMOylation can occur in both cytoplasm and nucleus on lysine residues. SUMOylation occurs as a modifier in s-amino group of lysine residues in target protein through a multi-enzymatic cascade. In this reaction, SUMO is connected to a lysine residue in substrate protein by covalent linkage via three enzymes, namely activating (E1), conjugating (E2) and ligase (E3). Often, SUMOylation modifications occur at a consensus motif WKxE (where W represents Lys, Ile, Val or Phe and X any amino acid). SUMOylation plays a major role in many basic cellular processes like transcription control, chromatin organization, accumulation of macromolecules in cells, regulation of gene expression and signal transduction and is necessary for the conservation of genome integrity.

Lipidation involves the covalent attachment of lipids to proteins. These PTMs may use a variety of lipids, including octanoic acid, myristic acid, palmitic acid, palmitoleic acid, stearic acid, cholesterol, etc. Myristoylation, palmitoylation and prenylation can be considered as the three main types of these lipid modifications. Palmitoylation is the covalent attachment of fatty acids, like palmitic acid on the Cys, Gly, Ser, Thr and Lys. S-palmitoylation contains a reversible covalent addition of a 16-carbon fatty acid chains, palmitate, to a cysteine via a thioester linkage. Palmitoyl-CoA (as the lipid substrate) is attached to the target protein. Mostly, S-palmitoylation occurs in eukaryotic cells and plays critical roles in many different biological processes including protein function regulation, protein-protein interaction, membrane-protein associations, neuronal development, signal transduction, apoptosis and mitosis.

Myristoylation (N-myristoylation) is an irreversible PTM that occurs mainly on cytoplasmic eukaryotic proteins. Myristoylation happens approximately in 0.5-1.5% of eukaryotic proteins. In myristoylation, after removal of the initiating Met, a 14-carbon saturated fatty acid, called myristic acid, is attached to the N-terminal glycine residue via a covalent bond. This attachment is often observed in Met-Gly-X-X-X- Ser/Thr motif and is catalyzed by an N-myristoyl transferase (NMT) (there are at least two types of NMT enzymes, NMT1 and NMT2, in humans). Proteins that undergo this PTM play critical roles in regulating the cellular structure and many biological processes such as stabilizing the protein structure maturation, signalling, extracellular communication, metabolism and regulation of the catalytic activity of the enzymes.

Prenylation is another important lipid-based PTM, which occurs after translation as an irreversible covalent linkage mainly in the cytosol. This reaction occurs on cysteine and near the carboxyl-terminal end of the substrate protein. Prenylation has two main forms: farnesylation and geranylation. These two forms contain the addition of two different types of isoprenoids to cysteine residues: farnesyl pyrophosphate (15-carbon) and geranylgeranyl pyrophosphates (20-carbon), respectively. In prenylated proteins, one can find a consensus motif at the C-terminal; the motif is CAAX where C is cysteine, A is an aliphatic amino acid and X is any amino acid. This process is catalyzed by three prenyltransferase enzymes: farnesyltransferase (FT) and two geranyl transferases (GT1 and GT2). Prenylation is known as a crucial physiological process for facilitating many cellular processes such as protein-protein interactions, endocytosis regulation, cell growth, differentiation, proliferation and protein trafficking.

N-sulfation or O-sulfation includes the addition of a negatively charged sulfate group by nitrogen or oxygen to an exposed residue on the target protein. Currently, PTS is observed mainly in secreted and transmembrane proteins in multicellular eukaryotes. This reaction is catalyzed by two transmembrane enzymes, tyrosyl protein sulfotransferases 1 and 2 (TPST1 and TPST2). TPSTs govern the transfer of an activated sulfate from 3-phospho adenosine 5-phosphosulfate to residues within acidic motifs of polypeptides.

The expressed protein may contain an AVI tag. The Avi-tag peptide (GLNDIFEAQKIEWHE) is recognized by BirA ligase which enzymatically attaches a biotin molecule to a single lysine residue within the Avi-tag sequence. The enzyme can be present during expression to attach the biotin to the expressed tag after expression of the POI.

Conditions for performing post-translational modifications can be screened in parallel. The expression composition may be assembled on the device from mixing a variety of droplets in order to screen a variety of compositions in parallel.

By way of example, the screening reagents may include,
Chaperone mix (e.g., PUREfrex GroE mix)
Kinase 1 (e.g., NEB CK2)
Kinase 2 (e.g., NEB PKA)
Protease 1 (e.g., NEB TEV)
Protease 2 (e.g., Merck HRV 3C)
N-acetyl transferase
Common metal ions cocktail
Common co-factors cocktail

The compositions can be blended by the user and the level of expression of the protein of interest monitored in each of the blended conditions.

Metal ions may include one or more of the following: MgCl₂, CuCl₂, ZnCl₂, CaCl₂, MnCl₂, NiCl₂, CoCl₂.

Co-factors may include one or more of Nicotinamide adenine dinucleotide (NAD), Flavin adenine dinucleotide (FAD), S-adenosyl methionine (SAM), pyridoxal phosphate (PLP) Co-enzyme A (CoA), thiamine pyrophosphate (TPP) or haem.

Chaperones may include one or more of DnaK, DnaJ, GrpE, heat shock proteins, protein disulfide isomerase (PDI), human protein disulfide isomerase (hPDI), disulfide bond C (DsbC), a thioredoxin, such as TRXB1, Caseinolytic peptidase B protein homolog (CLPB) or FK506 binding proteins (FKBPs).

The additive may be for example one or more reducing agents. The additive may be selected from DTT, glutathione (GSH) or glutathione disulfide (GSSG).

The added protease may cleave the protein of interest from flanking regions. The flanking regions mat include tags used for detection or solubility or other buffer regions. The flanking regions may be cleaved by any protease. The protease may be a serine, threonine, cysteine or methionine protease. The protease may be a metalloprotease. The protease may be a TEV or 3C protease.

The TEV protease may act upon the amino acid sequence ENLYFQS. The template for expression of the POI may include the nucleic acid sequence

GAGAACCTGTACTTCCAGAGC.

The 3C protease may act upon the amino acid sequence LEVLFQGP. The template for expression of the POI may include the nucleic acid sequence

CTCGAGGTTCTGTTCCAAGGACCT.

The method may be used to perform a surfactant screen to identify the best surfactant for expression of a particular protein. The surfactant may be ionic, nonionic, or zwitterionic.

Surfactant molecules are composed of a hydrophobic region and a hydrophilic region. This amphiphilic structure enables surfactants to obtain a discoidal conformation in the solution, known as micelles. Micelles solubilize membrane proteins by encompassing the transmembrane domains of integral membrane proteins, with the loops and hydrophilic regions exposed to solvent. The minimum concentration of a surfactant necessary to form micelles and extract membrane proteins is called critical micelle concentration or CMC.

Depending on the charge of hydrophilic group, surfactants are classified into three groups: ionic, nonionic, and zwitterionic surfactants. Ionic surfactants carry a charged group, either negative (anionic) or positive (cationic), and historically have been the most efficient group of detergents in extracting membrane proteins from lipid bilayers. However, ionic detergents can have deleterious effects on protein-protein interactions and often lead to protein denaturation. Sodium dodecyl sulfate (SDS) and sodium cholate are two common examples of ionic detergents.

Nonionic surfactants are currently the most popular and successful group of surfactants in solubilizing membrane proteins for both functional and structure determination purposes. This is due to their nondisruptive nature, which enables them to preserve the native structure of the target protein by breaking protein-lipid interactions instead of protein-protein interactions. Alkyl glycoside surfactants such as n-dodecyl-ß-D-maltoside (DDM), n-decyl-ß-D-maltoside (DM), n-Octyl-ß-D-Glucopyranoside (OG), and n-Nonyl-ß-D-Glucopyranoside (NG) by contributing to the purification and crystallization of about 70% of membrane proteins are the most common nonionic surfactants for protein studies. Another advantage of nonionic surfactants is that they do not interfere with optical measurements, which enables fluorescence-based experiments on expressed proteins.

Zwitterionic surfactants typically have an intermediate level of harshness between ionic and nonionic detergents. They carry both positive and negative charged groups in their polar regions with an overall net charge of zero. An example of a Zwitterionic surfactant is lauryldimethylamine-N-oxide or LDAO.

Membrane mimetic systems, such as nanodiscs and styrene malic acid lipid particles (SMALPs), provide an alternative platform for stabilization of membrane proteins and hence eliminate the deleterious effects of detergents on these macromolecules. Nanodiscs are composed of phospholipid patches surrounded by two copies of membrane scaffold protein (MSP), a genetically engineered version of human serum apolipoprotein A-I.

Any particular nucleic acid template can be expressed using the system described herein. Three types of nucleic acid templates used in CFPS include plasmids, linear expression templates (LETs), and mRNA. Plasmids are circular templates, which can be produced either in cells or synthetically. LETs can be made via PCR. While LETs are easier and faster to make, plasmid yields are usually higher in CFPS. mRNA can be produced through in-vitro transcription systems. The methods use a single nucleic acid template per droplet. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied (e.g. PURE-FREX). The PURE system is composed of all the enzymes that are involved in transcription and translation, as well as highly purified 70 S ribosomes. The protein synthesis reaction of the PURE system lacks proteases and ribonucleases, which are often present as undesired molecules in cell extracts.

Disclosed is a composition comprising a template nucleic acid, reagents for cell-free protein synthesis and one or more enzymes for post translationally covalently modifying the expressed folded protein. The modification changes the molecular weight of the expressed protein.

The term digital microfluidic device refers to a device having a two-dimensional array of planar microelectrodes. The term excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplet to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

Once the CFPS reagents have been enclosed in the droplets, additional reagents can be supplied by merging the original droplet with a second droplet. The second droplet can carry any desired additional reagents, including for example oxygen or ‘power’ sources, or test reagents to which it is desired to expose to the expressed protein.

The droplets can be aqueous droplets. The droplets can contain an oil immiscible organic solvent such as for example DMSO. The droplets can be a mixture of water and solvent, providing the droplets do not dissolve into the bulk oil.

The droplets can be in a bulk oil layer. A dry gaseous environment simply dries the bubbles onto the surface during the expression process, leaving comet type smears of dried material by evaporation. Thus the device is filled with liquid for the expression process. Alternatively, the aqueous droplets can be in a humidified gaseous environment. A device filled with air can be sealed and humidified in order to provide an environment that reduces evaporation of CFPS droplets.

The droplets containing the cell-free extract having the components for protein expression will therefore typically be in the oil filled environment before the nucleic acid templates are added to the droplets. The templates can be added by merging droplets on the microfluidic device. Alternatively, the templates can be added to the droplets outside the device and then flowed into the device for the expression process. For example the expression process can be initiated on the device by increasing the temperature. The expression system typically operates optimally at temperatures above standard room temperatures, for example at or above 29° C.

The expression process typically takes many hours. Thus the process should be left for at least 30 minutes or 1 hour, typically at least 2 hours. Expression can be left for at least 12 hours. During the process of expression the droplets should be moved within the device. The moving improves the process by mixing the reagents and ensuring sufficient oxygen is available within the droplet. The moving can be continuous, or can be repeated with intervening periods of non-movement.

Thus the aqueous droplet can be repeatedly moved for at least a period of 30 minutes or one hour whilst the protein is expressed. The aqueous droplet can be repeatedly moved for at least a period of two hours whilst the protein is expressed. The aqueous droplet can be repeatedly moved for at least a period of twelve hours whilst the protein is expressed. The act of moving the droplet allows mixing within the droplet, and allows oxygen or other reagents to be supplied to the droplet. The act of moving improves the level of protein expression over a droplet which remains static.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescense.

The droplet can be moved using any means of electrokinesis. The aqueous droplet can be moved using electrowetting-on-dielectric (EWoD). Electrowetting on a dielectric (EWoD) is a variant of the electrowetting phenomenon that is based on dielectric materials. During EWoD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWoD device can be delivered through segmented electrodes, active-matrix thin-film transistors or digital micromirrors. Optically-activated s-Si EWoD devices are well known in the art for actuating droplets (J. Adhes. Sci. Technol., 2012, 26, 1747-1771).

The oil in the device can be any water immiscible or hydrophobic liquid. The oil can be mineral oil, silicone oil, an alkyl-based solvent such as decane or dodecane, or a fluorinated oil. The air in the device can be any humidified gas.

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated. Alternatively the droplets can be presented in a humidified air filled device.

The droplet can be formed before entering the microfluidic device and flowed into the device.

Alternatively the droplets can be merged on the device. Included is a method comprising merging a first droplet containing a nucleic acid template such as a plasmid with a second droplet containing a cell-free system having the components for protein expression to form the droplet.

The droplets can be split on the device either before, during or after expression. Included herein is a method further comprising splitting the droplet into multiple droplets. If desired the split droplets can be screened with further additives. Included is a method wherein one of more of the split droplets are merged with additive droplets for screening.

Through an affinity tag, such as a FLAG-tag, HIS-tag, GST-tag, MBP-tag, STREP-tag, or other form of affinity tag, CFPS-expressed proteins can be immobilized to a solid-support affinity resin and fresh batches of CFPS reagent can be delivered over the said resin. Thus, renewed reagents can be used to carry out protein synthesis, closely mimicking industrial methods of continuous flow (CF) and continuous exchange (CE) CFPS. By mimicking CF-and CE-CFPS, users can scale up their CFPS production methods.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move

CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

Droplets can also contain additives to reduce the effects of biofouling on digital microfluidic surfaces. Specifically, droplets containing CFPS components can also contain additives such as surfactants or detergents to reduce the effects of biofouling on the hydrophobic or superhydrophobic surface of a digital microfluidic device (Langmuir 2011, 27, 13, 8586-8594). Such droplets may use antifouling additives such as TWEEN 20, Triton X-100, and/or Pluronic F127. Specifically, droplets containing CFPS components may contain TWEEN 20 at 0.1% v/v, Triton X-100 at 0.1% v/v, and/or Pluronic F127 at 0.08% w/v.

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting or otherwise adulterating the lysate results in a decrease in expression level of the protein of interest. Thus performing CFPS on DMF where the surfactants are added to the solutions being moved will necessarily result in a dilution and adulteration of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell-free system with a GFP₁₁ (or similar) peptide tag, it's downstream complementation with a GFP₁₋₁₀ (or similar) detector polypeptide is hindered in the presence of surfactant. Removal of the surfactant from the aqueous phase is therefore advantageous.

Rather than adding surfactants to the aqueous sample, it is instead possible to add surfactant, such as a sorbitan ester such as Span85 (e.g. Sorbitan trioleate, Sigma Aldrich, SKU 8401240025), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric or a mixture thereof. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the Split GFP (e.g. GFP₁₁/GFP₁₋₁₀) system, so removing surfactants from the reagent mix and instead adding them to the oil can be beneficial.

The peptide tag can be attached to the C or N terminus of the protein. The peptide tag may be one component of a green fluorescent protein (GFP). For example the peptide tag may be GFP₁₁ and the further polypeptide GFP_{1-10.} The peptide tag may be one component of sfCherry. The peptide tag may be sfCherryl₁ and the further polypeptide sfCherry₁₋₁₀. The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry₁₁ peptide tags and the synthesis occurs in the presence of multiple sfCherry1−10 polypeptides. The protein of interest may be fused to one or more sfCherryl1 peptide tags and one or more GFP₁₁ peptide tags and the synthesis occurs in the presence of one or more GFP₁₋₁₀ polypeptides and one or more sfCherry₁₋₁₀ polypeptides.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrokinesis occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c . {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWoD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/Σᵣ)^{1/2}. Thus, to reduce actuation voltage, it is required to reduce (t/Σᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at a 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWoD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminum oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μL or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference.

Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

Described herein is an electrokinetic device, including:
a first substrate having a matrix of electrodes, wherein each of the matrix electrodes is coupled to a thin film transistor, and wherein the matrix electrodes are overcoated with a functional coating comprising:

one or more dielectric layer(s) comprising silicon nitride, hafnium oxide or aluminum oxide in contact with the matrix electrodes,
a conformal layer comprising parylene in contact with the dielectric layer, and a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

The electrokinetic devices as described may be used with other elements, such as for example devices for heating and cooling the device or reagent cartridges for the introduction of reagents as needed.

### EXAMPLES

### 3C Protease Mixing at t=0.

expressing construct_1437

(SUMO_VEGF_3C_HIS_L3_CCGFP11V1_STOP_TERM_RBUF200)

ccGFP₁₋₁₀ detector supplied at 65.5 mg/ml

Print bioink 2 (PUREfrex reconstituted cell-free expression system)

3C protease (Sigma Aldrich) (Human Rhinovirus 3C Protease, Levlfqgp site protease, PreScission Protease).

### Methods

### Reaction Setup

1. Print bioink 2 30 μL and DNA 10 μL—Setup 2X print bioink 2 30 mL with EB 10 μL (10 mM HEPES+0.05% F127 pH 8 buffer)
2. Add 3C protease to the expression mix at final concentration of 0.083 mg/mL.
3. Incubate overnight at 29° C.
4. ccGFP1−10 detector complementation at a final concentration of 50 μM was carried out for 5 hours at 29° C.
5. Run a semi-native SDS-PAGE gel electrophoresis without boiling to keep the complemented ccGFP fluorescence. After taking the picture the gel was stained using Instantblue from abcam.

### Results

The samples were set up with construct_1437 in print bioink 2 containing 3C protease and 3C buffer as negative control for overnight expression. The samples were then complemented with ccGFP₁₋₁₀ detector for 5 hours. Next, the total fraction was set aside and the rest of the sample was spun down for 15 minutes at max. centrifuge speed to harvest soluble and insoluble fractions. After removing the soluble supernatant the insoluble pellet was washed and resolubilised in the TNG buffer.

3C activity showing protein cleavage on electrowetting device

3C protease cleavage on device with print bioink 2 expressing construct_1437 (SUMO_VEGF_3C_HIS_L3_CCGFP11V1_STOP_TERM_RBUF200

### Materials

ccGFP₁₋₁₀ detector supplied at 65.5 mg/mL

Print bioink 2—(PUREfrex reconstituted cell-free expression system)

3C protease (Sigma Aldrich) (Human Rhinovirus 3C Protease, Levlfqgp site protease, PreScission Protease).

### Methods

### Day 1

### Reaction Setup

Print bioink 2 30 μL and DNA 10 μL—Setup 2X, Print bioink 2 30 μL with EB 10 μL (10 mM HEPES+0.05% F127 pH 8 buffer)

### Day 2

1. ccGFP₁₋₁₀ detector complementation at a final concentration of 50 μM was carried out for 5 hours at 29° C.
2. Prepare 3C protease master mix in 3C reaction buffer
3. Prepare 25 mL 3C reaction Buffer

### 625 μL of HEPES

### 6.25 mL of NaCl

### 50 μL EDTA

25 μL DTT (1 M stock)

### Rest-MilliQ

4. 3C Reaction buffer containing 25 mM HEPES (pH 7.5), 150 mM NaCl, 1 mM EDTA, and 1 mM DTT.
5. Prepare 3C Master mix: 20 μL 3C Protease+20 μL Reaction buffer (0.5 μg/μL)
6. The reagents were loaded onto an electrowetting cartridge and mixed as required.
7. The remaining of both pre-mixes were incubated at 29° C. for one hour as positive and negative in-tube controls.
8. Droplets were retrieved from separate exit ports from the cartridge to avoid cross-contamination.
9. Perform a semi-native gel electrophoresis without boiling to keep the complemented ccGFP fluorescence. After taking the picture the gel was stained using Instantblue from abcam.

### Semi Native SDS-PAGE Gel With Fluorescence and Coomassie Stains

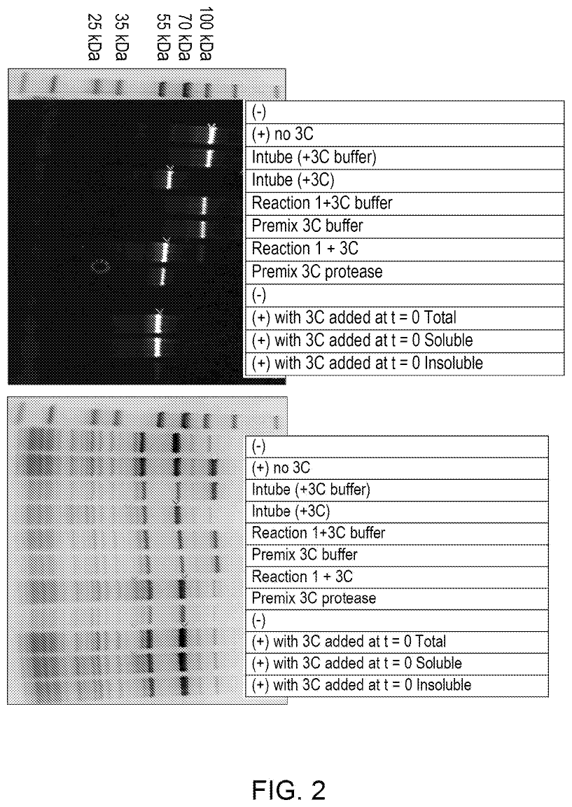

All the fractions from the tubes and cartridge (pre-mix/cassette mix, 3C buffer/3C) were retrieved from the device and run on a SDS-PAGE gel. The gel was imaged for fluorescence bands and then Coomassie stained. The images obtained showed no activity for all the negative controls and an almost full cleavage for all in-tube controls. The premixed sample with 3C loaded on device and the on-device mixed 3C sample showed 100% cleavage (FIG. 2). The data shows that 3C cleavage works when both premixed with 3C protease and mixed and incubated on cartridge with droplet mixing scripts. The protease acts to cleave expressed proteins when used on the electrowetting device, either when mixed on device in droplets or off device.

### Plate Reader

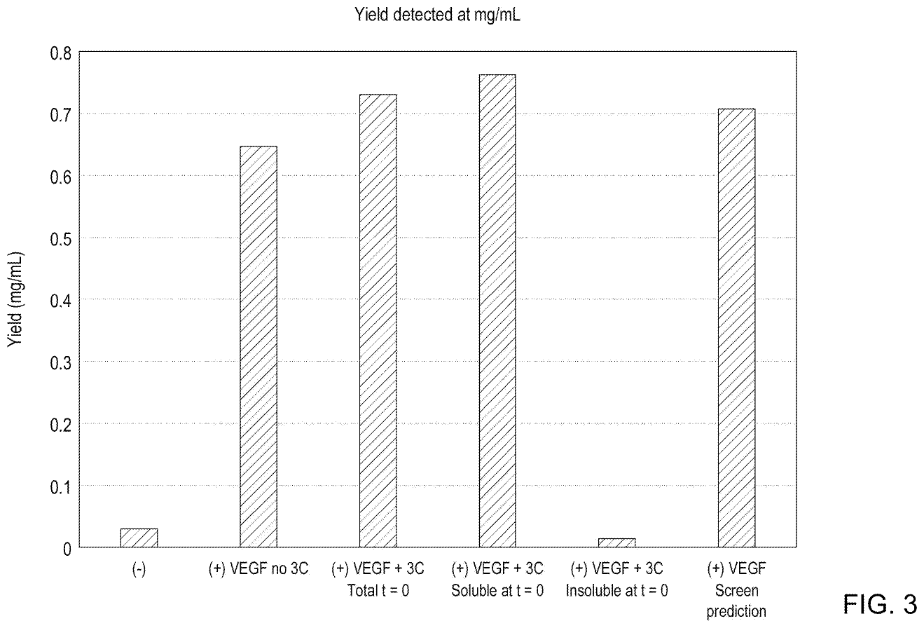

Fluorescence plate reader measurements indicated that the total protein detected in absence of 3C had lower fluorescent yield when the detector is bound to the protein VEGF with solubility tag. In the presence of 3C protease, when complemented after 3C cleavage of SUMO_VEGF, the total and soluble fraction were detected at higher yields. The analysis from the plate reader can be seen in FIG. 3.

The 3C protease was active in the expression reaction mixture and was able to act on the expressed full length VEGF protein in order to cleave the 3C site.

### On Device Cleavage Using 3C Protease

3C cleavage on electrowetting device using protein test panel.

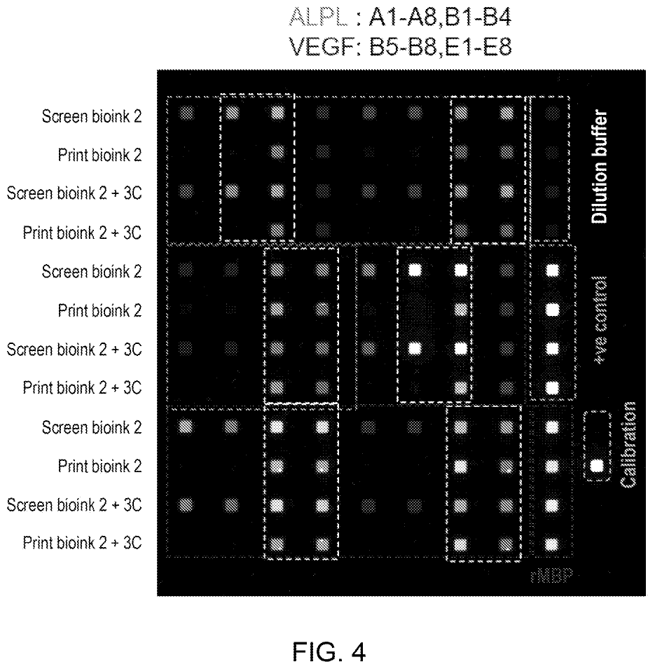

24 nucleic acid constructs as shown in FIG. 4 (12 construct with POI ALPL and 12 with POI VEGF) were mixed with Bioink 2 containing a reconstituted expression system (PUREfrex reconstituted cell-free expression system), either with or without a ccGFP₁₋₁₀ detector protein present. Each droplet contain an equivalent droplet containing 3C protease. Thus 96 droplets, along with expression controls were allowed to express for 10 h at 29° C. Each droplet was merged with a droplet containing ccGFP₁₋₁₀ detector protein and incubated for a further 5 hours. Results of the expression can be seen in FIG. 3. Some constructs expressed with a high level of signal. In some cases speckles are seen, indicating protein aggregation. In comparison with the expression data observed in the presence or absence of 3C, there was no significant difference observed in expression profile. The presence of 3C did not have a significant impact on the level of expression on the electrowetting device.

1. A method for expressing and changing the molecular weight of an expressed protein using a single reagent composition comprising a template nucleic acid, reagents for cell-free protein synthesis and one or more enzymes which post translationally covalently modify and amend the molecular weight of the expressed protein.

2. The method according to claim 1, wherein the enzyme performing the post translational modification is selected from glycosylating enzymes, proteases, redox active enzymes, acetyl transferases, phosphorylases, kinases, methylases, ubiquitinases, sumoylation, isoprenylases.

3. The method according to claim 1, wherein the enzyme performing the post translational modification is involved in the post-translational modification process for phosphorylation, methylation, sulfation, acetylation, ubiquitylation, prenylation, myristoylation, SUMOylation, palmitoylation or glycosylation.

4. The method according to claim 2, wherein the enzyme is a protease which cuts the expressed protein.

5. The method according to claim 4, wherein the protease is selected from a TEV protease or a 3C protease.

6. The method according to claim 2, or claim 3 wherein the enzyme covalently appends a chemical group to the expressed protein.

7. The method according to any one preceding claim, wherein the expression is performed in droplets on an electrowetting-on-dielectric (EWoD) device.

8. A method for the synthesis of a modified protein on an electrowetting-on-dielectric (EWoD) device, the method comprising taking a reaction system having at least one template nucleic acid encoding a protein of interest, blending droplets to form a series of droplets having varying cell-free reagent compositions, including enzymes for protein synthesis, the template nucleic acid and one or more enzymes for post translationally covalently modifying the expressed folded protein and monitoring the synthesis of the protein of interest in the various compositions, thereby identifying a composition suitable for expression and modification of the protein of interest.

9. The method according to any one of claims 1 to 8, wherein the reagent composition contains synthesized or isolated ribosomes, initiation factors, elongation factors, aminoacyl-tRNA synthetases, methionyl tRNA transformylases, tRNAs, amino acids, ribonucleoside triphosphates, 10-formyl 5,6,7,8-tetrahydrofolic acid (FD), salts, and water.

10. The method according to claim 9, wherein the reagent composition contains a polyethylene glycol, an allolactose, an aldohexose or a thiogalactopyranoside.

11. The method according to any one of claims 1 to 10, wherein the reagent composition contains buffers, surfactants, metal ions, chaperones, co-factors or additional protein components.

12. The method according to any one of claims 1 to 11, wherein the reagent composition contains a mixture of cell lysate and reconstituted expression systems.

13. The method according to any one of claims 1 to 12, wherein the cell lysate is derived from mammalian cells, prokaryotic cells, yeast cells, plant cells or insect cells.

14. The method according to any one preceding claim, wherein the reagent contains a termination factor.

15. The method according to any one preceding claim, wherein the reagent composition is formed on an EWoD device by merging a first droplet containing a cell lysate and a second droplet having one or more enzymes for post translationally covalently modifying the expressed folded protein.

16. The method according to any one preceding claim, wherein the reagent composition is formed on an EWoD device by merging a first droplet containing the template nucleic acid with a second droplet containing the reagent composition having enzymes for protein synthesis and a third droplet having one or more enzymes for post translationally covalently modifying the expressed folded protein.

17. A composition comprising a template nucleic acid, reagents for cell-free protein synthesis and one or more enzymes for post translationally covalently modifying the expressed folded protein.

18. The composition according to claim 17 comprising a template nucleic acid, reagents for cell-free protein synthesis and a protease which cuts the expressed protein.

19. The composition according to claim 18, wherein the protease is selected from a TEV 5 or 3C protease.

20. The composition according to any one of claims 17-19, wherein the composition is in droplets on an electrowetting-on-dielectric (EWoD) device.

21. The composition according to any one of claims 17-20, wherein expression system is a reconstituted expression system.
